(19) United States
(12) Patent Application Publication
(10) Pub. No.: US 2022/0249303 A1
(43) Pub. Date: Aug. 11, 2022

(54) REUSABLE SANITARY NAPKIN AND A COMBINED PANTY AND REUSABLE SANITARY NAPKIN UNDERWEAR

(71) Applicant: 3RTW INTERNATIONAL CORPORATION, Taichung City (TW)

(72) Inventor: Sheng-Chieh YANG, Taichung City (TW)

(21) Appl. No.: 17/583,307

(22) Filed: Jan. 25, 2022

(30) Foreign Application Priority Data
Feb. 4, 2021 (TW) 110104269
Jun. 1, 2021 (TW) 110119856

(51) Int. Cl. A61F 13/511; A61F 13/47; A61F 13/56; A61F 13/66; A61F 13/15

(52) U.S. Cl. CPC A61F 13/51121; A61F 13/47; A61F 2013/51139; A61F 13/665; A61F 13/15268; A61F 13/5616

(57) ABSTRACT

A reusable sanitary napkin includes an outer body, a first absorbent fabric component, and a skin-contacting fabric component. The outer body includes a base layer component and a top waterproof layer component. The top waterproof layer component is attached to a top surface of the base layer component, and has a through hole. The first absorbent fabric component is attached to the top surface of the base layer component, and is registered with the through hole. The skin-contacting fabric component is attached between the first absorbent fabric component and the top waterproof layer component. A boundary of the through hole is disposed on the skin-contacting fabric component.

### CROSS-REFERENCE TO RELATED APPLICATIONS

This application claims priorities of Taiwanese Invention Patent Application No. 110104269, filed on Feb. 4, 2021, and Taiwanese Invention Patent Application No. 110119856, filed on Jun. 1, 2021.

### FIELD

The disclosure relates to a sanitary napkin, more particularly to a reusable eco-friendly sanitary napkin and a combined panty and reusable sanitary napkin underwear.

### BACKGROUND

Sanitary napkins are women's daily necessities during menstruation, and most of these are disposable sanitary napkins. Although they are pretty convenient to use, their disposable nature means that over the years they have contributed to producing a lot of waste.

Given the environmental protection issues of disposable sanitary napkins, some manufactures have developed a fabric sanitary napkin that can be reused after washing. The fabric sanitary napkin is entirely made of fabric, and includes a top layer, an absorbent layer, and a base layer. However, in use, even if menstrual blood and secretions have penetrated the top layer and are absorbed by the absorption layer, the absorbed menstrual blood may ooze out, causing the skin-touching top layer to get wet and stick to the skin, making for an unpleasant user experience.

### SUMMARY

Therefore, an object of the disclosure is to provide a reusable eco-friendly sanitary napkin that can improve user comfort.

According to the disclosure, a reusable sanitary napkin includes an outer body, a first absorbent fabric component, and a skin-contacting fabric component. The outer body includes a base layer component and a top waterproof layer component. The base layer component has a top surface. The top waterproof layer component has a connection surface attached to the top surface of the base layer component, a skin-contacting surface opposite to the connection surface, and a through hole extending through the skin-contacting surface and the connection surface. The first absorbent fabric component is attached to the top surface of the base layer component, and is registered with the through hole. The skin-contacting fabric component is attached between the first absorbent fabric component and the connection surface of the top waterproof layer component. A boundary of the through hole is disposed on the skin-contacting fabric component.

Another object of the disclosure is to provide a combined panty and reusable sanitary napkin underwear that can improve the comfort of use.

According to the disclosure, the combined panty and reusable sanitary napkin underwear includes a panty and a reusable sanitary napkin. The panty has a crotch section adapted to be disposed between thighs of a wearer. The reusable sanitary napkin is connected to the crotch section of the panty, and includes an outer body, a first absorbent fabric component, and a skin-contacting fabric component. The outer body includes a base layer component and a top waterproof layer component. The base layer component has a top surface. The top waterproof layer component has a connection surface attached to the top surface of the base layer component, a skin-contacting surface opposite to the connection surface, and a through hole extending through the skin-contacting surface and the connection surface. The first absorbent fabric component is attached to the top surface of the base layer component, and is registered with the through hole. The skin-contacting fabric component is attached between the first absorbent fabric component and the connection surface of the top waterproof layer component, and covers an underside of the through hole, such that a boundary of the through hole is disposed on the skin-contacting fabric component.

### BRIEF DESCRIPTION OF THE DRAWINGS

Other features and advantages of the disclosure will become apparent in the following detailed description of the embodiments with reference to the accompanying drawings, of which:

FIG. 1 is a perspective view of a reusable sanitary napkin of a first embodiment of a combined panty and reusable sanitary napkin underwear according to the disclosure;

FIG. 2 is an exploded perspective view of the sanitary napkin of the first embodiment with a fastening unit being omitted;

FIG. 3 is a cross-sectional view taken along line III-III in FIG. 1;

FIG. 4 is a fragmentary enlarged view of FIG. 3;

FIG. 5 is a side view of the first embodiment;

FIG. 6 is a photograph representing a side-by-side comparison of the embodiment according to this disclosure on the right and a conventional product on the left;

FIG. 7 is a perspective view of a reusable sanitary napkin of a second embodiment of the combined panty and reusable sanitary napkin underwear according to the disclosure;

FIG. 9 is an exploded perspective view of the second embodiment;

FIG. 9 is a cross-sectional view of the reusable sanitary napkin of the second embodiment;

FIG. 10 is a partly exploded perspective view of a third embodiment of the combined panty and reusable sanitary napkin underwear according to the disclosure;

FIG. 11 is a partly exploded perspective view of a fourth embodiment of the combined panty and reusable sanitary napkin underwear according to the disclosure;

FIG. 12 is a cross-sectional view of the fourth embodiment;

FIG. 13 is a perspective view of a fifth embodiment of the combined panty and reusable sanitary napkin underwear according to the disclosure; and

FIG. 14 is a cross-sectional view of the fifth embodiment.

### DETAILED DESCRIPTION

Before the present invention is described in greater detail, it should be noted that where considered appropriate, reference numerals or terminal portions of reference numerals have been repeated among the figures to indicate corresponding or analogous elements, which may optionally have similar characteristics.

The present disclosure will be described with embodiments in detail below by reference to the drawings. Note that the relative position terminology used in the following description as “front,” “rear,” “left,” “right,” “top,” and “bottom” are based on the orientation shown in each figure and the normal use orientation.

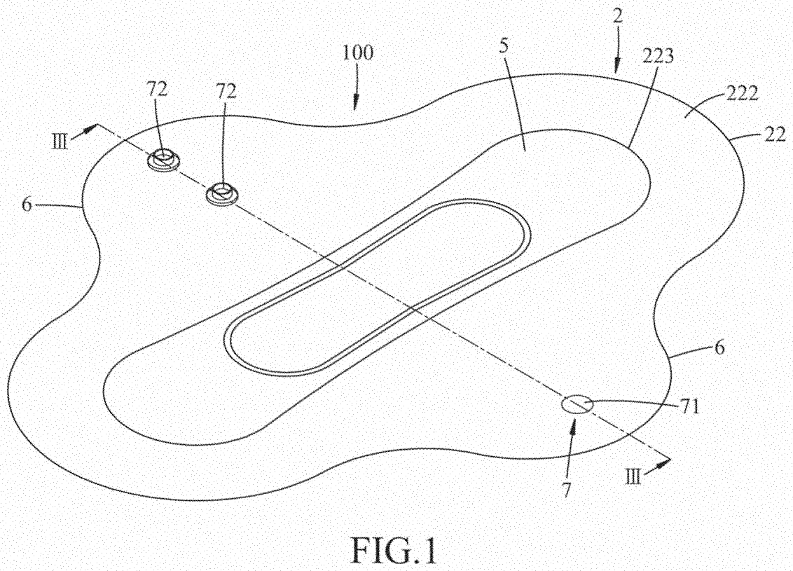

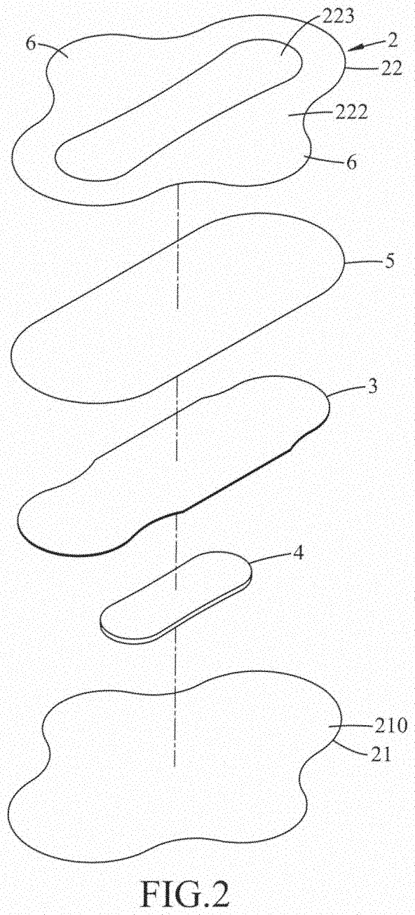

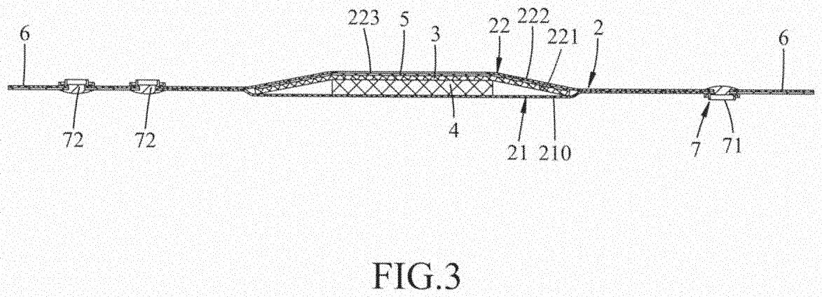

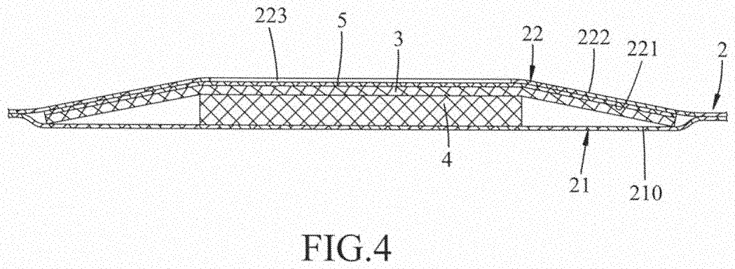

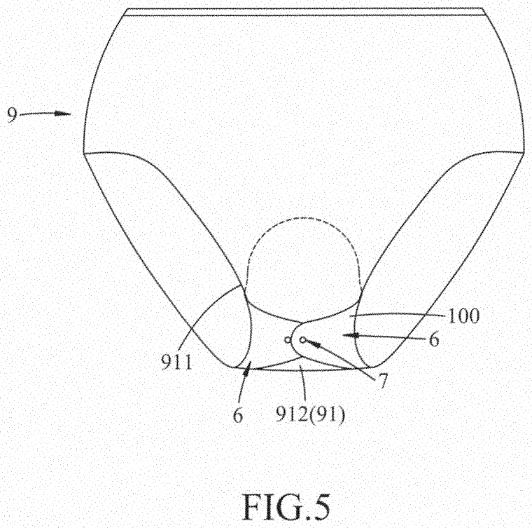

As shown in FIGS. 1 to 5, the first embodiment of a combined panty and reusable sanitary napkin includes a panty 9 and a reusable sanitary napkin 100. The panty 9 has a crotch portion 91 that is adapted to be disposed between thighs of a wearer, and that has a proximate surface 911 facing the wearer's body and a distal surface 912 opposite to the proximate surface 911. The reusable sanitary napkin 100 includes an outer body 2, a first absorbent fabric component 3, a second absorbent fabric component 4, a skin-contacting fabric component 5, two wings 6, and a fastening unit 7.

The outer body 2 includes a base layer component 21 and a top waterproof layer component 22. The base layer component 21 has a top surface 210. The top waterproof layer component 22 has a connection surface 221 that is attached to the top surface 230 of the base layer component 21, a skin-contacting surface 222 that is opposite to the connection surface 221, and a through hole 223 that extends through the skin-contacting surface 222 and the connection surface 221. The through hole 223 is smaller than the first absorbent fabric 3. It is noted that the top waterproof layer component 22 covers a portion of the skin-contacting fabric component 5 except the portion of the skin-contacting fabric component 5 corresponding to the through hole 223, and it can reduce the amount of menstrual blood that is absorbed in the reusable sanitary napkin 100 oozing out to the skin-contacting surface 222. The top waterproof layer component 22 of the present disclosure may be made of a fiber fabric laminated with a waterproof layer or a fiber fabric coated with a waterproof coating.

The first absorbent fabric component 3 is attached to the top surface 210 of the base layer component 21, and is registered with the through hole 223.

The second absorbent fabric component 4 is attached between the base layer component 21 and the skin-contacting fabric component 5, and is aligned with the through hole 223. The second absorbent fabric component 4 is smaller than the first absorbent fabric component 3. In this embodiment, the second absorbent fabric component 4 is attached to the bottom aide of the first absorbent fabric component 3, and is located between the first absorbent fabric component 3 and the base layer component 21. In other embodiments, the second absorbent fabric component 4 may be alternatively attached to the top side of the first absorbent fabric component 3 and is located between the first absorbent fabric component 3 and the skin-contacting fabric component 5.

In this embodiment, each of the first absorbent fabric component 3 and the second absorbent fabric component 4 is made of a material selected from a group consisting of woven fabrics, non-woven fabrics, and a combination thereof.

The skin-contacting fabric component 5 is positioned between the first absorbent fabric component 3 and the connection surface 221 of the top waterproof layer component 22, and is attached to the connection surface 221 of the top waterproof layer component 22. The skin-contacting fabric component 5 covers an underside of the through hole 223. As such, a boundary of the through hole 223 is disposed on the skin-contacting fabric component 5, such that fluid which passes through the through hole 223 will flow through the skin-contacting fabric component 5 into the first absorbent fabric component 3. In this embodiment, the skin-contacting fabric component 5 has a plurality of pores (not shown) to allow menstrual blood to pass through quickly and be absorbed by the first absorbent fabric component 3 and the second absorbent fabric component 4. It should be noted that if the skin-contacting fabric component 5 is made of a relatively hydrophilic material, the pores will be smaller, and if the skin-contacting fabric component 5 is made of a relatively hydrophobic material, the pores will be larger.

The wings 6 are connected respectively to opposite lateral sides of the outer body 2. The fastening unit 7 is connected to the wings 6 for interconnecting the wings 6.

When the two wings 6 are folded over the crotch section 91 of the panty 9 toward each other, they can be detachably linked by the fastening unit 7 to thereby secure the reusable sanitary napkin 100 onto the panty 9. In this embodiment, the fastening unit 7 includes a male component 71 that is disposed on one of the wings 6, and two female components 72 that are disposed on the other one of the wings 6. The wearer can connect one of the two female components 72 to the male component 71, depending on her needs. It should be noted that the fastening unit 7 does not have to be in the form of male and female components 71 and 72; hook-and-loop fasteners or other fastening connections may also be used.

To connect the reusable sanitary napkin 100 and the panty 9 together, the reusable sanitary napkin 100 is first placed on the proximate surface 911 of the crotch section 91 of the panty 9. Then, the two wings 6 are folded around the crotch section 91 to the distal surface 912 of the crotch section 91, and the male component 71 is coupled to the selected one of the female components 72 to secure the reusable sanitary napkin 100 on the panty 9. In use, the through hole 223 of the top waterproof layer component 22 is positioned adjacent to the private parts of the wearer in the use position. When the wearer menstruates, the blood first flows through the skin-contacting fabric component 5, then it is absorbed by the first absorbent fabric component 3 and the second absorbent fabric component 4. The skin-contacting fabric component 5 prevents the first absorbent fabric component 3 and the second absorbent fabric component 4 from coming into direct contact with the wearer's skin, and allows menstrual blood to pass through quickly and be absorbed by the first absorbent fabric 3 and the second absorbent fabric 4, preventing the wearer's skin from becoming sticky and uncomfortable.

In this embodiment, the second absorbent fabric component 4 has a higher absorbency than the first absorbent fabric component 3. The first absorbent fabric component 3 has a higher absorbency than the skin-contacting fabric component 5. The aforementioned arrangement can direct menstrual blood downwards, preventing it from pooling on the skin-contacting fabric 5. In addition, since the first absorbent fabric component 3 is registered with the through hole 223, menstrual blood and secretions will flow directly through the first absorbent fabric component 3, and into the second absorbent fabric component 4. Since the first absorbent fabric component 3 is covered and will not come into directly contact with the wearer's skin, the wearer will feel more comfortable. Furthermore, in addition to having a higher absorbency than the first absorbent fabric component 3, the second absorbent fabric component 4 is also thicker, which further enhances the absorption of menstrual blood and secretions and improves the comfort of use.

The base layer component 21 is made of a waterproof material, and can prevent menstrual blood from leaking therethrough. The waterproof base layer component 21 is easy to clean after usage.

It should be noted that, the components of the reusable sanitary napkin 100 may be joined together using heat compression bonding, ultrasonic welding, high-frequency welding, sewing, and so on.

In summary, the reusable sanitary napkin 100 of the embodiment can quickly absorb menstrual blood and secretions through the arrangement of the skin-contacting fabric component 5, the second absorbent fabric component 4, and the first absorbent fabric component 3. The top waterproof layer component 22 can limit the area of the skin-contacting fabric component 5 that comes into direct contact with the wearer's skin and prevent menstrual blood from oozing out to the skin-contacting surface 222 of the top waterproof layer component 22, thereby improving the comfort of use.

It is noted that although the reusable sanitary napkin 100 in this embodiment is exemplified to include the outer body 2, the first absorbent fabric component 3, the second absorbent fabric component 4, and the skin-contacting fabric component 5, the reusable sanitary napkin 300 may include only the outer body 2 and the first absorbent fabric component 3 in other embodiments, with the second absorbent fabric component 4, the skin-contacting fabric component 5, and the fastening unit 7 being omitted.

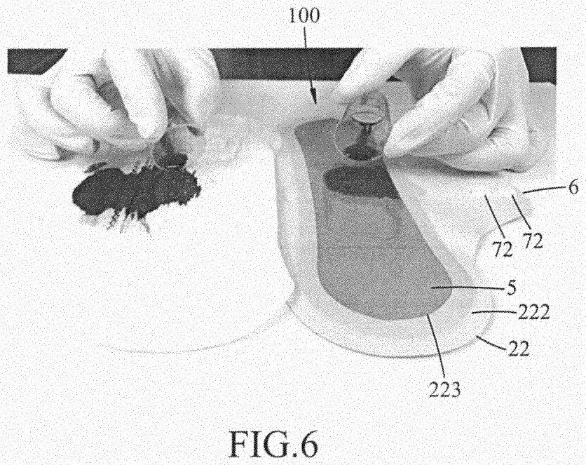

FIG. 6 shows a production example of the reusable sanitary napkin 100, on the right side, and a conventional sanitary napkin on the left side. It can be observed that the diffusion area of the liquid on the skin-contacting fabric component 5 of the reusable sanitary napkin 100 is much smaller than that of the conventional sanitary napkin when both have been poured with the same amount of liquid. The reason for this is that the liquid can be absorbed by the first absorbent fabric component 3 after it flows into the skin-contacting fabric component 5; however, the layer in the conventional sanitary napkin that is in direct contact with the wearer's body is made of a liquid absorbent material, so that the liquid is absorbed and diffused to a greater extent. As a result, the present reusable environmentally friendly sanitary napkin 100 is expected to have enhanced user comfort.

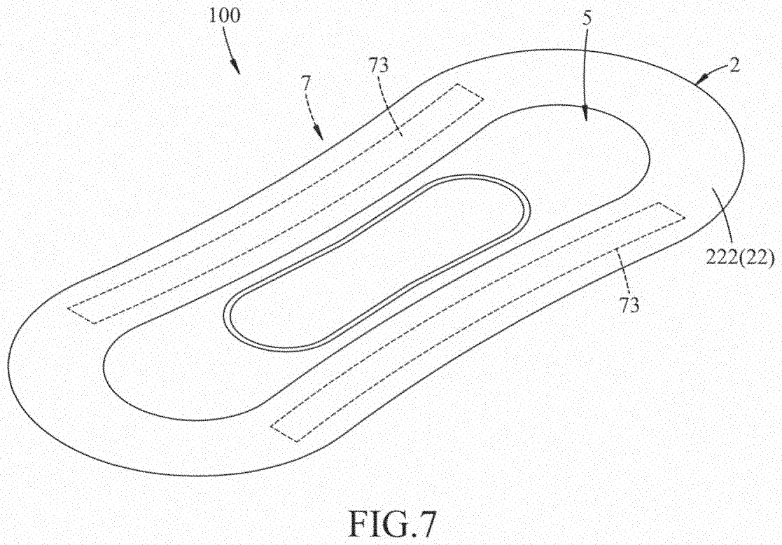

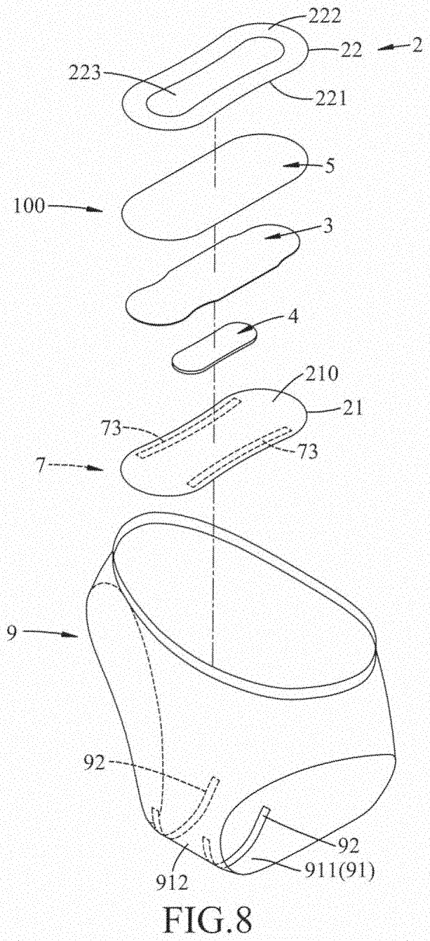

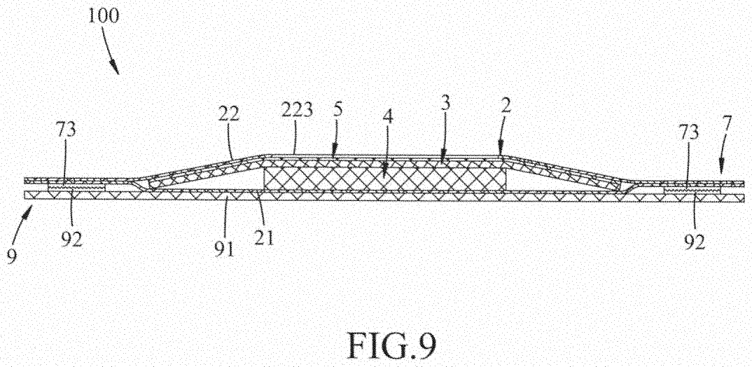

As shown in FIGS. 7 to 9, a second embodiment of the combined panty and reusable sanitary napkin underwear has a structure similar to that of the first embodiment. The main differences between this embodiment and the previous embodiment resides in the following. In this embodiment, the panty 9 further has two panty-fastening components 92 that are positioned on the proximate surface 911 of the crotch section 91. The location of the panty-fastening components 92 may vary in other embodiments.

In this embodiment, the wings 6, which are illustrated in the previous embodiment, are omitted. The width between lateral sides of the base layer component 21 and the width between the lateral sides of the top waterproof layer component 22 correspond to the width of the crotch section 91 of the panty 9.

In this embodiment, the male and female components 71, 72, which are illustrated in the previous embodiment, are omitted. The fastening unit 7 includes two napkin-fastening components 73 disposed on a bottom side of the base layer component 21. The two napkin fastening components 73 are positioned respectively on the left and right sides of the through hole 223, and are respectively and detachably coupled to the panty-fastening components 92 of the panty 9.

In this embodiment, the panty-fastening components 92 of the panty 9 and the napkin-fastening components 73 are configured as a book-and-loop structure, but the type of fastening connection therebetween may vary in other embodiments.

There is no need for additional wings 6 for connection with the panty 9 because the reusable sanitary napkin 100 is detachably coupled to the panty 9 via the connection between the panty-fastening components 92 of the panty 9 and the napkin-fastening components 73. Furthermore, the reusable sanitary napkin 100 when coupled to the panty 9 is securely attached and will not move in the front-to-back direction or deflect to the left or right relative to the panty 9, thereby preventing side leaking.

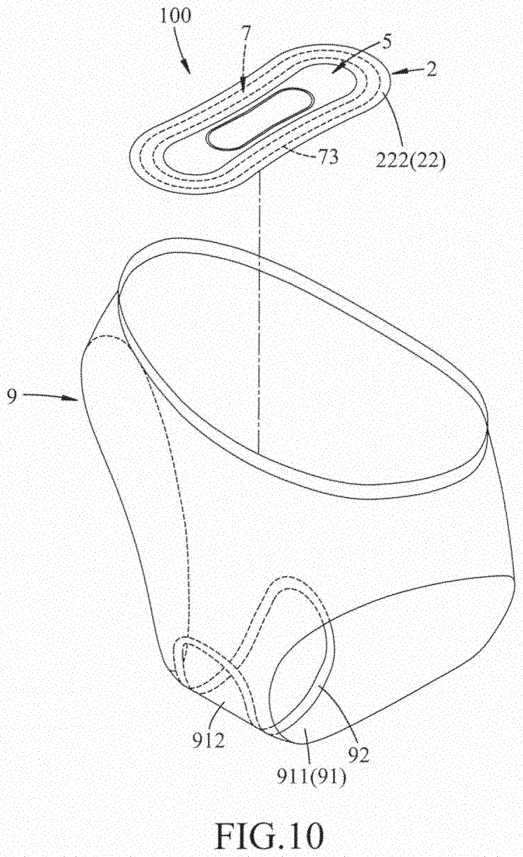

Referring to FIG. 10, a third embodiment of the combined panty and reusable sanitary napkin underwear has a structure similar to that of the second embodiment. The main difference between this embodiment and the second embodiment resides in the following.

The fastening unit 7 includes just one napkin fastening component 73 which is ring-shaped and surrounds the through hole 223. The panty 9 has only one panty-fastening component 92 that is ring-shaped and that is coupled detachably to the napkin-fastening component 73. The third embodiment can achieve the same purpose and effect as the second embodiment.

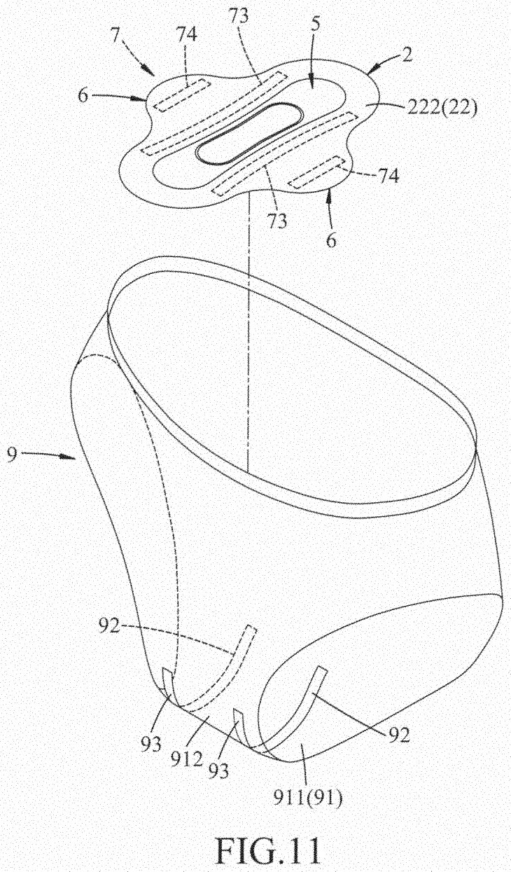

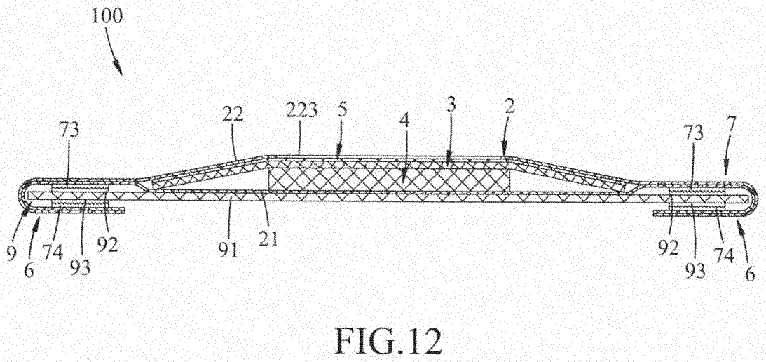

Referring to FIGS. 11 and 12, a fourth embodiment of the combined panty and reusable sanitary napkin underwear has a structure similar to that of the second embodiment. The main difference between this embodiment and the second embodiment resides in the following.

In this embodiment, the panty 9 further has two reinforcing components 93 arranged on the distal surface 912 of the crotch section 91.

The reusable sanitary napkin 100 has the wings 6. The fastening unit 7 further includes two seizing components 74, which are located respectively on the wings 6. In this embodiment, the reinforcing components 93 of the panty 9 and the seizing component 74 of the fastening unit 7 are configured as a hook-and-loop structure and are detachably coupled together.

To couple the reusable sanitary napkin 100 to the panty 9, the reusable sanitary napkin 100 is first placed in the panty 9, with the outer body 2 adjacent to the proximate surface 911 of the crotch section 91, and the napkin-fastening components 73 are then coupled to the panty fastening components 92. Afterwards, the wings 6 are folded toward each other over the distal surface 912 of the panty 9 to, couple respectively the seizing components 74 with the reinforcing components 93. The connection between the seizing components 74 and the reinforcing components 93 strengthens the attachment of the reusable sanitary napkin 100 to the panty 9. It can also keep the reusable sanitary napkin 100 from moving on the panty 9 and prevent side leakage.

Furthermore, because the napkin-fastening components 73 are connected to the panty-fastening components 92 of the panty 9, the reusable sanitary napkin 100 can stay in place even if the seizing component 74 on the wings 6 falls off or the wings 6 fail to connect with the crotch section 91 for other reasons.

In this embodiment, the wings 6 are folded over the distal surface 912 with a gap therebetween instead of overlapping each other as illustrated in the first embodiment. As a result, the total thickness of the reusable sanitary napkin 100 and the crotch section 91 of the panty 9 can be reduced, resulting in a less observable outline on the outer garment (not shown) when worn. The fourth embodiment can also achieve the same purpose and effect as the second embodiment.

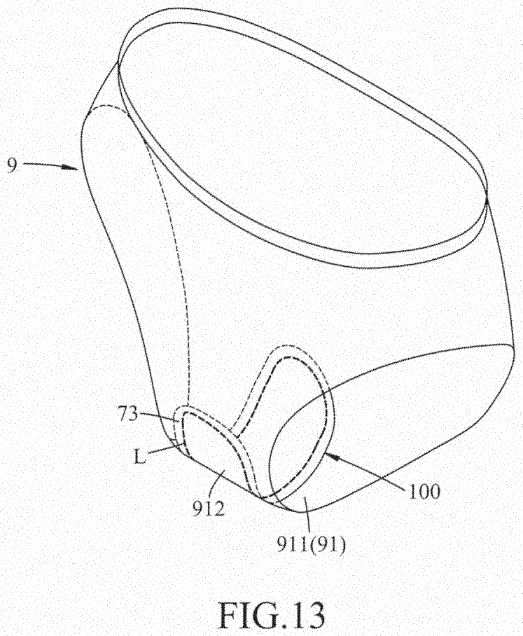

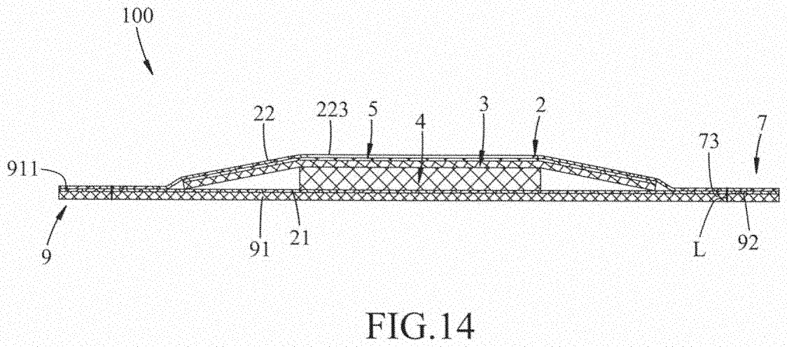

Referring to FIGS. 13 and 14, a fifth embodiment has a structure similar to that of the third embodiment. The main difference between this embodiment and the third embodiment resides in the following.

In this embodiment, the napkin-fastening component 73 of the fastening unit 7 is connected to the panty-fastening component 92 of the panty 9 by sewing along a sewing line (L).

By connecting the napkin-fastening component 73 to the panty-fastening component 92 through sewing, the connection strength between the reusable sanitary napkin 100 and the panty 9 can be improved. The fifth embodiment can also achieve the same purpose as the third embodiment.

In summary, with the skin-contacting fabric component 5 being covered with the top waterproof layer component 22, the area of the skin-contacting fabric component 5 that directly contacts the wearer's skin can be reduced. This arrangement will prevent menstrual blood from oozing out onto the skin-contacting surface 222 of the top waterproof layer component 22, hence boosting user comfort and achieving the purpose of the present disclosure.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A reusable sanitary napkin comprising:
an outer body including
a base layer component that has a top surface, and
a top waterproof layer component that has a connection surface attached to said top surface of said base layer component, a skin-contacting surface opposite to said connection surface, and a through hole extending through said skin-contacting surface and said connection surface;
a first absorbent fabric component attached to said top surface of said base layer component, and being registered with said through hole; and
a skin-contacting fabric component attached between said first absorbent fabric component and said connection surface of said top waterproof layer component and covering an underside of said through hole, such that a boundary of said through hole being disposed on said skin-contacting fabric component.

2. The reusable sanitary napkin as claimed in claim 1, wherein said through hole is smaller than said first absorbent fabric component.

3. The reusable sanitary napkin as claimed in claim 1, further comprising two wings connected respectively to opposite lateral sides of said outer body, and a fastening unit connected to said wings for interconnecting said wings.

4. The reusable sanitary napkin as claimed in claim 1, wherein said first absorbent fabric component is made of a material selected from a group consisting of woven fabrics, non-woven fabrics, and a combination thereof.

5. The reusable sanitary napkin as claimed in claim 1, further comprising a second absorbent fabric component attached between said base layer component and said skin-contacting fabric component, and being smaller than said first absorbent fabric component.

6. The reusable sanitary napkin as claimed in claim 5, wherein said second absorbent fabric component is made of a material selected from a group consisting of woven fabrics, non-woven fabrics, and a combination thereof.

7. The reusable sanitary napkin as claimed in claim 5, wherein said second absorbent fabric component is registered with said through hole, and is attached to one of top and bottom sides of said first absorbent fabric component.

8. The reusable sanitary napkin as claimed in claim 7, wherein said top waterproof layer component is made of fiber fabric laminated with a waterproof layer.

9. The reusable sanitary napkin as claimed in claim 1, further comprising at least one napkin-fastening component that is disposed on a bottom side of said base layer component, and that is adapted to be disposed on a crotch section of a panty and coupled to at least one panty-fastening component mounted on the crotch section.

10. The reusable sanitary napkin as claimed in claim 9, wherein said at least one napkin-fastening component is adapted to be detachably coupled to the at least one panty-fastening component of the panty.

11. A combined panty and reusable sanitary napkin underwear, comprising:
a panty having a crotch section adapted to be disposed between thighs of a wearer; and
a reusable sanitary napkin connected to said crotch section of said panty, and including
an outer body that includes
a base layer component having a top surface, and
a top waterproof layer component having a connection surface that is attached to said top surface of said base layer component, a skin-contacting surface that is opposite to said connection surface, and a through hole that extends through said skin-contacting surface and said connection surface,
a first absorbent fabric component that is attached to said top surface of said base layer component, and that is registered with said through hole, and
a skin-contacting fabric component that is attached between said first absorbent fabric component and said connection surface of said top waterproof layer component such that fluid which passes through said through hole flows through said skin-contacting fabric component into said first absorbent fabric component.

12. The combined panty and reusable sanitary napkin underwear as claimed in claim 11, wherein said through hole is smaller than said first absorbent fabric component.

13. The combined panty and reusable sanitary napkin underwear as claimed in claim 11, wherein said reusable sanitary napkin further includes a second absorbent fabric component attached between said base layer component and said skin-contacting fabric component, and being smaller than said first absorbent fabric component.

14. The combined panty and reusable sanitary napkin underwear as claimed in claim 11, wherein:
said panty further has at least one panty-fastening component mounted on said crotch section; and
said reusable sanitary napkin further includes at least one napkin-fastening component disposed on a bottom side of said base layer component and coupled to said at least one panty-fastening component.

15. The combined party and reusable sanitary napkin underwear as claimed in claim 14, wherein said at least one napkin-fastening component of said reusable sanitary napkin is detachably coupled to said at least one panty-fastening component of said panty.
